(19) United States
(12) Patent Application Publication
(10) Pub. No.: US 2026/0010723 A1
(43) Pub. Date: Jan. 8, 2026

(54) Classification of Psychiatric Disorder Related Spontaneous Communication Using Large Language Model Embeddings

(71) Applicant: Bristol-Myers Squibb Company, Princeton, NJ (US)

(72) Inventors: Ryan Allen Shewcraft, Stamford, CT (US); Mariann Micsinai Balan, Lawrenceville, NJ (US); John Carter Schwarz, New Hope, PA (US)

(73) Assignee: Bristol-Myers Squibb Company, Princeton, NJ (US)

(21) Appl. No.: 19/258,144

(22) Filed: Jul. 2, 2025

Related U.S. Application Data
(60) Provisional application No. 63/668,361, filed on Jul. 8, 2024.

(51) Int. Cl. G06F 40/289; A61B 5/00; A61B 5/16; G06F 40/40

(52) U.S. Cl. CPC G06F 40/289; A61B 5/16; A61B 5/165; A61B 5/168; A61B 5/4803; G06F 40/40

(57) ABSTRACT

A method includes obtaining a corpus of input text including a plurality of textual utterance sets. Each textual utterance set from the plurality of textual utterance sets includes a respective set of multiple textual utterances originating from a respective one of multiple disorders/conditions. For each corresponding textual utterance: the method also includes processing, using a large language model (LLM), the corresponding textual utterance to generate a respective LLM embedding that represents the corresponding textual utterance in a high dimensional embedding space and processing, using a classification model, the respective LLM embedding to predict a classification label for the corresponding textual utterance, the predicted classification label including one of the multiple disorders/conditions. The method also includes training the classification model based on the classification label predicted for each corresponding textual utterance and the corresponding disorder/condition label paired with each corresponding textual utterance.

### CROSS-REFERENCE TO RELATED APPLICATIONS

This U.S. patent application claims priority under 35 U.S.C. § 119 (e) to U.S. Provisional Application 63/668,361, filed on Jul. 8, 2024. The disclosure of this prior application is considered part of the disclosure of this application and is hereby incorporated by reference in its entirety. Substitute

### TECHNICAL FIELD

This disclosure relates to classification of psychiatric disorder related spontaneous communication using large language model (LLM) embeddings.

### BACKGROUND

Psychiatric disorders encompass a diverse range of conditions affecting an individual's thoughts, emotions, and behaviors. These disorders are characterized by complex and heterogeneous symptomatology, making it difficult to establish precise diagnostic criteria and monitor disease progression over time. While standardized clinical interviews and questionnaires are used, they rely on subjective assessments and can be time-consuming or insensitive to subtle changes leading to potential misdiagnosis and delayed intervention.

Language, as a fundamental aspect of human communication, reflects the intricate interplay between thoughts, emotions, and experiences. Quantitative analysis of language usage has emerged as a valuable tool for providing objective measures for diagnosing and differentiating between different psychiatric disorders. Studies have shown that language-based features, such as syntactic complexity, semantic coherence, and emotional valence, can serve as reliable markers for differentiating between psychiatric disorders. For instance, individuals with schizophrenia often exhibit disturbances in their speech patterns, characterized by disorganized syntax and impaired semantic coherence. Similarly, individuals with borderline personality disorder have higher levels of overall expressive language impairment, as well as decreased syntactic and lexical complexity.

### DESCRIPTION OF DRAWINGS

FIG. 1 is a schematic view of a system for predicting psychiatric classification labels from textual utterances.

FIG. 2 is an example table depicting posts originating from individuals that identify as having a respective psychiatric disorder/condition from a plurality of common psychiatric disorders/conditions.

FIG. 3 is a plot of a two-dimensional visualization representing embeddings projected onto a high-dimensional embedding pace.

FIG. 4 is an example plot of receiver operating characteristic curves and area under the curve values.

FIG. 5 is a multi-class confusion matrix based on classification labels predicted by the system of FIG. 1.

FIG. 6 is a flowchart of an example arrangement of operations for a method of predicting classification labels.

FIG. 7 is a schematic view of an example computing device that may be used to implement the systems and methods described herein.

Like reference symbols in the various drawings indicate like elements.

### SUMMARY

One aspect of the disclosure provides a computer-implemented method that when executed on data processing hardware causes the data processing hardware to perform operations that include obtaining a corpus of input text including a plurality of textual utterance sets. Here, each textual utterance set from the plurality of textual utterance sets includes a respective set of multiple textual utterances originating from a respective one of multiple disorders/conditions. For each corresponding textual utterance from each respective textual utterance set: the operations also include processing, using a large language model (LLM), the corresponding textual utterance to generate a respective LLM embedding that represents the corresponding textual utterance in a high dimensional embedding space and processing, using a classification model, the respective LLM embedding to predict a classification label for the corresponding textual utterance, the predicted psychiatric classification label including one of the multiple disorders/conditions. The corresponding textual utterance is paired with a corresponding disorder/condition label indicating the respective disorder/condition from which the corresponding textual utterance originated. The operations also include training the classification model based on the classification label predicted for each corresponding textual utterance and the corresponding disorder/condition label paired with each corresponding textual utterance.

Implementations of the disclosure may include one or more of the following optional features. In some implementations, the operations also include receiving regular expression codes indicating a list of terms related to the multiple disorders/conditions;

and for each respective textual utterance set: inspecting, using the regular expression codes, the textual utterances from the respective set of multiple textual utterances to identify any textual utterances containing one or more expressions related to the respective disorder/condition among the multiple disorders/conditions from which the respective set of multiple textual utterances originated; and for each corresponding textual utterance in the respective set of multiple textual utterances identified as containing any of the one or more expressions related to the respective disorder/condition, modifying or removing the corresponding textual utterance from the respective textual utterance set.

In some examples, each textual utterance from each respective set of multiple textual utterances include one or more sentences. Additionally or alternatively, each textual utterance from each respective set of multiple textual utterances may include a post from an individual to an online forum topic associated with the respective disorder/condition. In some additional examples, the multiple disorders/conditions include multiple common psychiatric disorders/conditions. For instance, the multiple common psychiatric disorders/conditions may include schizophrenia, borderline personality disorder, depression, attention deficit hyperactivity disorder, anxiety, post-traumatic stress disorder, and bipolar disorder.

In some implementations, the operations also include receiving frequency information associated with the number of textual utterances in each respective textual utterance set. Here, training the classification model is further based on the frequency information. In these implementations, the classification model may be trained based on the frequency information by assigning weights to the respective LLM embeddings generated for the textual utterances in each respective textual utterance set that are that are inversely proportional to the number of the textual utterances in each respective textual utterance set.

The LLM may be trained using representational instruction tuning. For instance, the LLM may be further trained using generative instruction tuning. The LLM may include a plurality of sliding window attention (SWA) layers. The LLM may include a plurality of grouped-query attention (GQA) layers.

In some examples, when processing the corresponding textual utterance to generate the respective LLM embedding, the LLM employs bidirectional attention followed by mean pooling of a final hidden state to generate the respective LLM embedding. The classification model may include a multiclass classifier that employs extreme gradient boosting.

In some implementations, the operations further include generating a two-dimensional visualization that represents the LLM embeddings projected in a two-dimensional embedding space. In some additional implementations, the operations also include, after training the classification model: receiving a textual input of one or more sentences from an individual; processing, using the LLM, the textual input to generate a respective LLM embedding that represents the textual input in the high dimensional embedding space; and processing, using the trained classification model, the respective LLM embedding to predict a classification label for the textual input. In these implementations, the textual input is not paired with any disorder/condition label that indicates the respective disorder/condition associated with the corresponding textual utterance.

Another aspect of the disclosure provides a system that includes data processing hardware and memory hardware storing instructions that when executed on the data processing hardware causes the data processing hardware to perform operations. The operations include obtaining a corpus of input text including a plurality of textual utterance sets. Here, each textual utterance set from the plurality of textual utterance sets includes a respective set of multiple textual utterances originating from a respective one of multiple disorders/conditions. For each corresponding textual utterance from each respective textual utterance set: the operations also include processing, using a large language model (LLM), the corresponding textual utterance to generate a respective LLM embedding that represents the corresponding textual utterance in a high dimensional embedding space and processing, using a classification model, the respective LLM embedding to predict a classification label for the corresponding textual utterance, the predicted psychiatric classification label including one of the multiple disorders/conditions. The corresponding textual utterance is paired with a corresponding disorder/condition label indicating the respective disorder/condition from which the corresponding textual utterance originated. The operations also include training the classification model based on the classification label predicted for each corresponding textual utterance and the corresponding disorder/condition label paired with each corresponding textual utterance.

This aspect of the disclosure may include one or more of the following optional features. In some implementations, the operations also include receiving regular expression codes indicating a list of terms related to the multiple disorders/conditions; and for each respective textual utterance set: inspecting, using the regular expression codes, the textual utterances from the respective set of multiple textual utterances to identify any textual utterances containing one or more expressions related to the respective disorder/condition among the multiple disorders/conditions from which the respective set of multiple textual utterances originated; and for each corresponding textual utterance in the respective set of multiple textual utterances identified as containing any of the one or more expressions related to the respective disorder/condition, modifying or removing the corresponding textual utterance from the respective textual utterance set.

In some examples, each textual utterance from each respective set of multiple textual utterances include one or more sentences. Additionally or alternatively, each textual utterance from each respective set of multiple textual utterances may include a post from an individual to an online forum topic associated with the respective disorder/condition. In some additional examples, the multiple disorders/conditions include multiple common psychiatric disorders/conditions. For instance, the multiple common psychiatric disorders/conditions may include schizophrenia, borderline personality disorder, depression, attention deficit hyperactivity disorder, anxiety, post-traumatic stress disorder, and bipolar disorder.

In some implementations, the operations also include receiving frequency information associated with the number of textual utterances in each respective textual utterance set. Here, training the classification model is further based on the frequency information. In these implementations, the classification model may be trained based on the frequency information by assigning weights to the respective LLM embeddings generated for the textual utterances in each respective textual utterance set that are that are inversely proportional to the number of the textual utterances in each respective textual utterance set.

The LLM may be trained using representational instruction tuning. For instance, the LLM may be further trained using generative instruction tuning. The LLM may include a plurality of sliding window attention (SWA) layers. The LLM may include a plurality of grouped-query attention (GQA) layers.

In some examples, when processing the corresponding textual utterance to generate the respective LLM embedding, the LLM employs bidirectional attention followed by mean pooling of a final hidden state to generate the respective LLM embedding. The classification model may include a multiclass classifier that employs extreme gradient boosting.

In some implementations, the operations further include generating a two-dimensional visualization that represents the LLM embeddings projected in a two-dimensional embedding space. In some additional implementations, the operations also include, after training the classification model: receiving a textual input of one or more sentences from an individual; processing, using the LLM, the textual input to generate a respective LLM embedding that represents the textual input in the high dimensional embedding space; and processing, using the trained classification model, the respective LLM embedding to predict a classification label for the textual input. In these implementations, the textual input is not paired with any disorder/condition label that indicates the respective disorder/condition associated with the corresponding textual utterance.

The details of one or more implementations of the disclosure are set forth in the accompanying drawings and the description below. Other aspects, features, and advantages will be apparent from the description and drawings, and from the claims.

### DETAILED DESCRIPTION

Psychiatric disorders encompass a diverse range of conditions affecting an individual's thoughts, emotions, and behaviors. These disorders are characterized by complex and heterogeneous symptomatology, making it difficult to establish precise diagnostic criteria and monitor disease progression over time. While standardized clinical interviews and questionnaires are used, they rely on subjective assessments and can be time-consuming or insensitive to subtle changes leading to potential misdiagnosis and delayed intervention.

Language, as a fundamental aspect of human communication, reflects the intricate interplay between thoughts, emotions, and experiences. Quantitative analysis of language usage has emerged as a valuable tool for providing objective measures for diagnosing and differentiating between different psychiatric disorders. Studies have shown that language-based features, such as syntactic complexity, semantic coherence, and emotional valence, can serve as reliable markers for differentiating between psychiatric disorders. For instance, individuals with schizophrenia often exhibit disturbances in their speech patterns, characterized by disorganized syntax and impaired semantic coherence. Similarly, individuals with borderline personality disorder have higher levels of overall expressive language impairment, as well as decreased syntactic and lexical complexity.

Furthermore, quantitative analysis of language usage can aid in tracking disease progression and treatment response. Longitudinal studies have demonstrated that changes in linguistic patterns over time can be indicative of disease progression and treatment outcomes. For example, changes in language usage have been shown to correlate with changes in current depression symptoms. Additionally, “tentativeness”, as measured by a higher degree of uncertainty reflected in use of language by individuals with anxiety, is correlated with quantitative levels of symptoms measure by the General Anxiety Disorder-7 (GAD-7) scale. These findings underscore the potential of quantitative language analysis as a sensitive and objective measure for monitoring disease trajectories and treatment efficacy.

Recent advancements in large language models (LLMs) have opened up exciting possibilities for quantitative assessment of neurological diseases. As a result of their multi-head attention mechanism architecture, LLMs project strings of text (sentences, paragraphs, etc.) onto a high dimensional embedding space which represents the semantic and syntactic relationships between words and phrases. In this embedding space, linguistically similar texts are likely to be geometrically co-located with one another. Based on the notion that differences in patterns of speech by individuals across psychiatric disorders, implementations are directed toward using an LLM to generate embeddings from utterances conveying spontaneous use of language such that the generated embeddings will occupy diagnosis-specific subspaces within the LLM high dimensional embedding space. In some examples, the LLM includes a Generative Representational Instruction Tuning (GRIT) LLM that includes a LLM trained to both handle, and distinguish between, generative and embedding tasks by using both representational instruction tuning and generative instruction tuning during training. In some examples, the GRIT LLM includes seven (7) billion parameters.

More specifically, implementations are directed toward utilizing the embeddings derived/generated by the LLM from the utterances to classify/label the utterances associated with a plurality of common psychiatric disorders/conditions. While the present disclosure classifies/labels the utterances associated with schizophrenia, borderline personality disorder, depression, attention deficit hyperactivity disorder (ADHD), anxiety, post-traumatic stress disorder (PTSD) and bipolar disorder, the present disclosure is not so limiting and can be adapted to classify the utterances as originating from fewer, additional, and/or one or more different psychiatric or neurological disorders/conditions than those previously mentioned. For predicting classification labels of the different psychiatric or neurological disorders/conditions, implementations herein leverage a multiclass classifier model with a softmax objective function to simultaneously predict the classification labels from the utterances associated with the plurality of common psychiatric or neurological disorders/conditions. Implementations may be adapted to leverage a binary classifier model to determine whether or not an utterance is associated with a particular neurological disorder/condition. As used herein, the utterances from which the classifier model predicts classification labels for may correspond to textual utterances input (e.g., typed) as well as spoken utterances that are subsequently transcribed into text. As will become apparent, the utterances may originate from individuals that are formally diagnosed with the common psychiatric or neurological disorders/conditions, as well as from individuals where knowledge of a formal diagnosis is unknown but there is a strong likelihood that the individuals self-identify with the common psychiatric or neurological disorders/conditions or are otherwise associated with someone having the common psychiatric or neurological disorders/conditions.

As will become apparent, the use of LLMs offers a novel approach to analyzing patterns of language usage from spontaneous, patient-generated communication. In the field of psychiatric disorders, the accurate analyzation of language usage has the potential to revolutionize the way psychiatric disorders are diagnosed and monitored. By analyzing the spontaneous use of language in online discussion data, valuable insights into the linguistic patterns that distinguish between different psychiatric disorders can be gained.

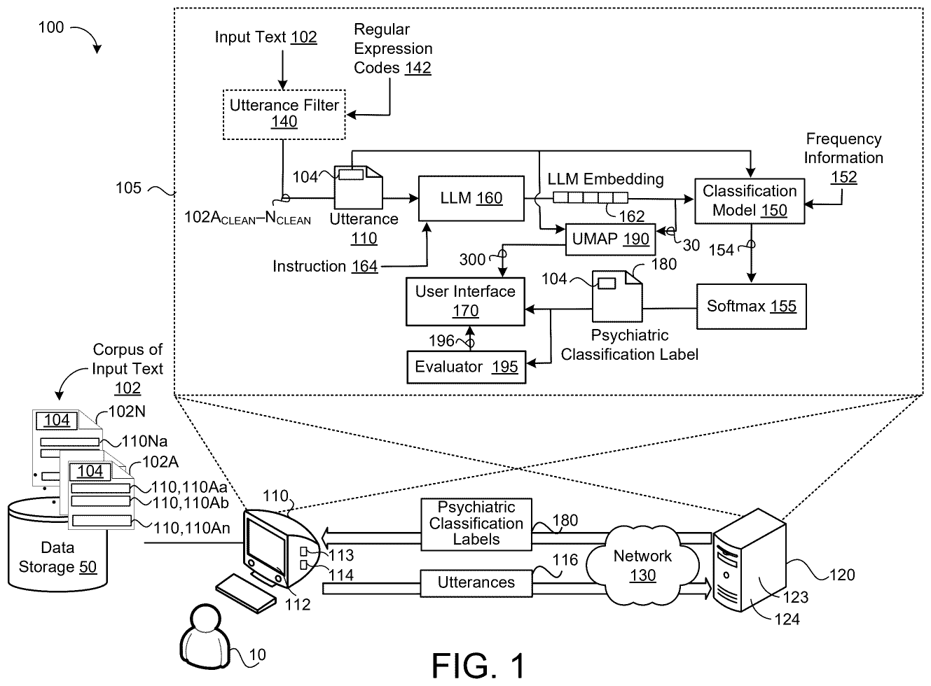

FIG. 1 illustrates an example system 100 for predicting psychiatric classification labels 180 from a corpus of input text 102 originating from user utterances 110 pertaining to a plurality of psychiatric or neurological conditions by leveraging a large language model (LLM) 160 that is trained using both representational instruction tuning and generative instruction tuning. Notably, by leveraging the LLM 160 trained using both representational instruction tuning and generative instruction tuning, the LLM is able perform both generative and embedding tasks. As used herein, the plurality of psychiatric conditions classified by the system 100 includes the following seven psychiatric disorders: schizophrenia; borderline personality disorder; depression; attention deficit hyperactivity disorder (ADHD); anxiety; post-traumatic stress disorder (PTSD); and bipolar disorder. However, the techniques disclosed herein for leveraging the LLM 160 to predict the psychiatric classification labels 180 are similarly applicable to predicting other types of psychiatric conditions and/or even particular diseases where embeddings generated by the LLM 160 from utterances by individuals associated with a particular disease occupy disorder/disease-specific subspaces within a LLM high dimensional embedding space 30. Notably, embeddings generated by the LLM 160 from utterances may convey changes in linguistic patterns over time to provide correlations indicative of disease progression and treatment outcomes.

A psychiatric disorder classification application (or simply ‘application’) 105 may execute on a user device 110 associated with a user 10 (e.g., a healthcare provider or a clinical trial designer) to enable the user 10 and the LLM 160 to interact with one another. The application 105 may access various components for facilitating the interaction between the user 10 and the LLM 160 in a natural manner. For instance, through the use of application programming interfaces (APIs) or other types of plug-ins, the application 105 may access an utterance cleaner 140, the LLM 160, a classification model 150 and associated Softmax 155, and a user interface 170.

The system 100 includes the user device 110, a remote computing system 120, and a network 130. The user device 110 includes data processing hardware 113 and memory hardware 114. The user device 110 may be any computing device capable of communicating with the remote computing system 120 through the network 130. The user device 110 includes, but is not limited to, desktop computing devices and mobile computing devices, such as laptops, tablets, smart phones, smart speakers/displays, digital assistant devices, smart appliances, internet-of-things (IoT) devices, infotainment systems, vehicle infotainment systems, and wearable computing devices (e.g., headsets, smart glasses, and/or watches).

The remote computing system 120 may be a distributed system (e.g., a cloud computing environment) having scalable elastic resources. The resources include computing resources 123 (e.g., data processing hardware) and/or storage resources 124 (e.g., memory hardware). Additionally or alternatively, the remote computing system 120 may be a centralized system. The network 130 may be wired, wireless, or a combination thereof, and may include private networks and/or public networks, such as the Internet.

The components leveraged by the psychiatric disorder classification application 105 may execute on the data processing hardware 113 of the user device 110 or on the data processing hardware 123 of the remote computing system 120. In some implementations, the components leveraged by the application 105 executes on both the data processing hardware 113 of the user device 110 and the data processing hardware 123 of the remote computing system 120. For instance, one or more components of the application 105 may execute on the data processing hardware 113 of the user device 110 while one or more other components of the application 105 may execute on the remote computing system 120.

The user 10 uses the application 105 for leveraging the LLM 160 to generate/derive embeddings 162 from textual utterances 110 and leveraging the classification model 150 and associated objective Softmax function 155 for predicting classification labels 180 of the different psychiatric disorders/conditions based on the embeddings 162 derived/generated by the LLM 160 from the textual utterances 110 originating from the plurality of common psychiatric disorders/conditions. The user device 110 may access data storage 50 that stores a corpus of input text 102 that includes a plurality of textual utterance sets 102A-N each including a respective set of multiple textual utterances 110 originating from a respective one of the psychiatric disorders/conditions among the plurality of common psychiatric disorders/conditions. Each textual utterance set 102A-N and/or each textual utterance 110 in the respective textual utterance set 102A-N may be paired with a psychiatric disorder/condition label 104 indicating the respective psychiatric disorder/condition the textual utterances 110 in the respective textual utterance set 102A-N originated from. For instance, a first textual utterance set 102A may include a respective set of multiple textual utterances 110Aa-An that originate from individuals having schizophrenia, a second textual utterance set 102B may include a respective set of multiple textual utterances 110Ba-Bn that originate from individuals having borderline personality disorder, a third textual utterance set 102C may include a respective set of multiple textual utterances 110Ca-Cn that originate from individuals having depression, a fourth textual utterance set 102D may include a respective set of multiple textual utterances 110Da-Dn that originate from individuals having ADHD, a fifth textual utterance set 102E may include a respective set of multiple textual utterances 110Ea-En that originate from individuals having anxiety, a sixth textual utterance set 102F may include a respective set of multiple textual utterances 110Fa-Fn that originate from individuals having PTSD, and a seventh textual utterance set 102N may include a respective set of multiple textual utterances 110Na-Nn that originate from individuals having bipolar disorder.

Each textual utterance 110 of the multiple textual utterances 110 in each textual utterance set 102A-N may include a post (e.g., an online forum post) by a corresponding individual that contains a phrase, a sentence, or multiple sentences. For instance, each textual utterance 110 may pertain to a respective online forum post in a corresponding subreddit class related to the respective psychiatric disorder/condition. Here, the first textual utterance set 102A may include textual utterances 110 each pertaining to a respective post by an individual to the subreddit class for schizophrenia: r/schizophrenia, the second textual utterance set 102B may include textual utterances 110 each pertaining to a respective post by an individual to the subreddit class for borderline personality disorder: r/bpd, the third textual utterance set 102C may include textual utterances 110 each pertaining to a respective post by an individual to the subreddit class for depression: r/depression, the fourth textual utterance set 102D may include textual utterances 110 each pertaining to a respective post by an individual to the subreddit class for ADHD: r/adhd, the fifth textual utterance set 102E may include textual utterances 110 each pertaining to a respective post by an individual to the subreddit class for anxiety: r/anxiety, the sixth textual utterance set 102F may include textual utterances 110 each pertaining to a respective post by an individual to the subreddit class for PTSD: r/ptsd, and the seventh textual utterance set 102N may include textual utterances 110 each pertaining to a respective post by an individual to the subreddit class for bipolar disorder: r/bipolarreddit.

With continued reference to FIG. 1, each textual utterance 110 from each textual utterance set 102A-N may initially pass through an optional utterance cleaner 140 configured to remove textual utterances 110 that contain text that exhibits one or more terms that are revealing of the respective subreddit class related to the respective psychiatric disorder/condition the classification model 150 is tasked to predict. More specifically, the utterance cleaner 140 inspects the textual utterance 110 from each respective textual utterance set 102A-N to identify corresponding textual utterances 110 that include regular expressions directly related to the title of the respective subreddit class. As such, the utterance cleaner 140 is configured to remove any textual utterances that are identified as including the regular expressions directly related to the title of the respective subreddit class. Alternatively, the utterance cleaner 140 may be configured to merely modify those textual utterances identified as including expressions directly related to the title by obfuscating text conveying those expressions or removing only a single sentence from the textual utterance that contains those expressions without removing other sentences in the textual utterance that do not contain the expressions. In some examples, the utterance cleaner 140 receives regular expression codes 140 indicating a list of terms for the subreddit classes that are each related to a respective one of the psychiatric disorders/conditions. Table 1 below shows example regular expression codes 142 that the utterance filter 140 may use to remove textual utterances 110 that would otherwise bias the LLM embeddings 162 generated/derived by the LLM 160, and thus, bias the classification model 150 in predicting psychiatric classification labels 180 for those textual utterances 110 that are revealing.

TABLE 1

| Subreddit | Regex terms for cleaning |
|---|---|
| r/adhd | adhd\|attention\|hyperact |
| r/anxiety | anxiety |
| r/bipolarreddit | bipolar |
| r/bpd | borderline\|bpd |
| r/depression | depress |
| r/pstd | ptsd\|post-traumatic\|post traumatic |
| r/schizophrenia | schiz |

Notably, in examples where the optional utterance cleaner 140 is implemented by the application 105, the utterance cleaner 140 is configured to provide respective cleaned textual utterance sets 102A_{CLEAN}-N_{CLEAN} such that each cleaned textual utterance set omits those textual utterances 110 identified by the utterance cleaner 140 as containing terms that match the regular expression code(s) 142 for the subreddit class related to the respective psychiatric disorder/condition. The utterance cleaner 140 may optionally be employed during training of the classification model 150 and removed during inference when the trained classification model 150 is employed to classify an input utterance.

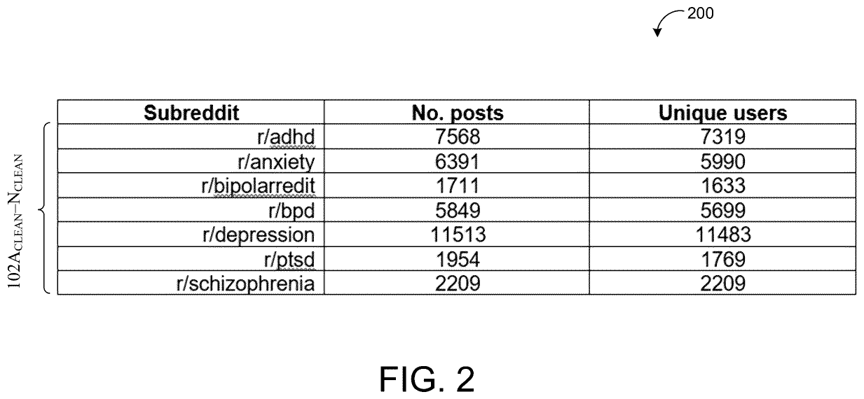

FIG. 2 shows a table 200 depicting a column of the subreddit classes each associated with a posts originating from individuals that identify as having a respective psychiatric disorder/condition from the plurality of common psychiatric disorders/conditions. As such, each row in the table 200 corresponds to a respective cleaned textual utterance set 102A_{CLEAN}-N_{CLEAN} from the corpus of input text 102 post cleaning by the utterance cleaner using the regular expression codes 142. The table 200 further includes a column that indicates a number of posts/textual utterances 110 in the respective textual utterance set 102A_{CLEAN}-N_{CLEAN} for each subreddit class as well as another column indicating a number of unique users. Thus, following the removal of posts/utterances 110 containing terms that would be revealing of the subreddit classes related to the psychiatric disorders/conditions the classification model 150 is configured to predict, there is nearly a seven-fold difference between the total number of posts in each subreddit class. For instance, the r-depression subreddit class has the greatest number of posts with 11,513 from 11,483 unique users (i.e., the respective third textual utterance set 102C_{CLEAN} includes 11,513 textual utterances 110), while the r-bipolarreddit has the least number of posts with 1,711 from 1,633 unique users (i.e., the respective seventh textual utterance set 102N_{CLEAN} includes 1,711 textual utterances 110. Notably, 36,102 out of 37,195, or 97.1-percent (97.1%), of the posts were made by unique users. Two users made five posts, seven users made four posts, and 54 users made three posts. The remaining 36,093 users, or 99.9-percent (99.8%), made only one or two posts. Furthermore, no user made posts in more than one subreddit class.

Referring back to FIG. 1, in other configurations, each textual utterance set 102A-N includes textual utterances expressed by one or more patients diagnosed with a respective psychiatric disorder/condition or disease that were tasked to provide samples of the textual utterances. In some scenarios, a first textual utterance set 102A includes textual utterances expressed by one or more patients diagnosed with a particular disorder/condition/disease and not undergoing treatment and a second textual utterance set 102B includes textual utterances expressed by the same or different one or more patients also diagnosed with the same particular disorder/condition/disease but have undergone treatment for treating the particular disorder/condition/disease. As such, the classification labels 180 predicted by the classification model 150 from the LLM embeddings 162 derived from textual utterances 110 in each respective textual utterance set may be correlated with disease progression and/or treatment outcomes, thereby providing an objective measure for monitoring disease trajectories and treatment efficacy.

The data storage 50 may be stored on memory hardware 114 of the user device 110 or on memory hardware 114 of the remote computing device 120 or server in communication with the user device 110. In some examples, at least one textual utterance 110 includes a transcription of one or more sentences derived from audio data characterizing an utterance spoken by a respective individual. Here, the spoken utterance may be recorded by the user device 110 or another device associated with the individual and converted into audio data, whereby an automatic speech recognition (ASR) system (not shown) may process the audio data characterizing the spoken utterance of the one or more sentences to generate the transcription corresponding to the textual utterance 110. Thus, while all textual utterances 110 in the input text corpus 102 inherently encompass linguistic details, such as syntactic information pertaining to the structure of sentences and semantic information related to the meaning conveyed by those structures, textual utterances 110 transcribed from speech may be annotated with additional metadata that may assist in the prediction of the classification labels 180 for those textual utterances 110. For instance, the ASR system may have the capability to capture metadata associated with a rate at which an individual spoke an utterance, a cadence of each term spoken in the utterance, or timestamps indicating where each term/word in the utterance began and/or ended. Metadata associated with acoustic information may also be derived from the audio data characterizing a spoken utterance to convey speaking style or prosodic details of the spoken utterance. For instance, the metadata may include pitch information, energy information, and/or duration of each word, or even syllable, in the one or more sentences of the spoken utterance. Accordingly, textual utterances 110 that were transcribed from speech with any of the aforementioned metadata may be annotated with the metadata and the metadata may be concatenated with LLM embeddings 162 produced by the LLM 160 for the utterances 110 and input to the classification model 150 for improving the prediction of the classification labels 180 for those utterances 110.

With continued reference to FIG. 1, the LLM 160 is configured to process each textual utterance 110 from each respective cleaned textual utterance set 102A_{CLEAN}-N_{CLEAN} and generate the respective LLM embedding 162 that represents the corresponding textual utterance 110 in a high dimensional embedding space. As such, the LLM embeddings 162 generated for all the textual utterances 110 in each respective cleaned textual utterance set 102A_{CLEAN}-N_{CLEAN} may be projected onto the high dimensional embedding space to represent the semantic and syntactic relationships between words and phrases of the textual utterances 110 amongst the cleaned textual utterance sets.

The LLM 160 may power the psychiatric disorder classification application 105 to provide personal chat bot capabilities for facilitating dialog conversations with the user 10 in natural language and performing tasks/actions on the user's behalf. The LLM 160 may leverage grouped-query attention (GQA) and sliding window attention (SWA) attention layers. GQA significantly accelerates inference speed while reducing memory requirements during decoding, while SWA permits the processing the longer input text sequences more effectively at a reduced computational cost, thereby alleviating a common limitation of convention LLMs.

In some examples, the LLM 160 includes a Generative Representational Instruction Tuning (GRIT) LLM that includes a LLM trained to handle both, and distinguish between, generative and embedding tasks by using both representational instruction tuning and generative instruction tuning during training. Generative instruction tuning includes training the LLM 160 to respond to instructions by generating an answer, while representation instruction tuning includes training the LLM 160 to represent a provided input according to an instruction. Via the instructions and separate loss functions, the GRIT LLM 160 learns to differentiate between generative and embedding tasks. The GRIT LLM 160 may use a batch size of 2,048 for embedding data when using representational instruction tuning and a batch size of 256 for generative data when using generative instruction tuning. In some examples, the GRIT LLM includes seven (7) billion parameters.

During inference, the LLM 160 may be prompted with an instruction 164 specifying a task for the LLM 160 to perform. For instance, when the LLM 160 is trained using both representational instruction tuning and generative instruction tuning, the instruction 164 may specify that the LLM 160 is to perform an embedding task such that the LLM 160 generates the LLM embedding 162 from the textual utterance 110 provided as input to the LLM 160. Similarly, the instruction 164 may instruct the LLM 160 to perform a generative task on input text in some scenarios.

In some implementations, for embedding tasks, the LLM 160 employs bidirectional attention followed by mean pooling of a final hidden state for generating a final representation, i.e., an LLM embedding 162 output from the LLM 160 for an utterance 110 that may be represented in the high dimensional embedding space 30. The LLM 160 may execute on the data processing hardware 113, 123 that includes a single graphical processing unit (GPU) web services instance. In some examples, the LLM 160 is trained using only representational instruction tuning to enable the LLM 160 to generate LLM embeddings 162 from textual utterances 110 provided as input to the LLM 160.

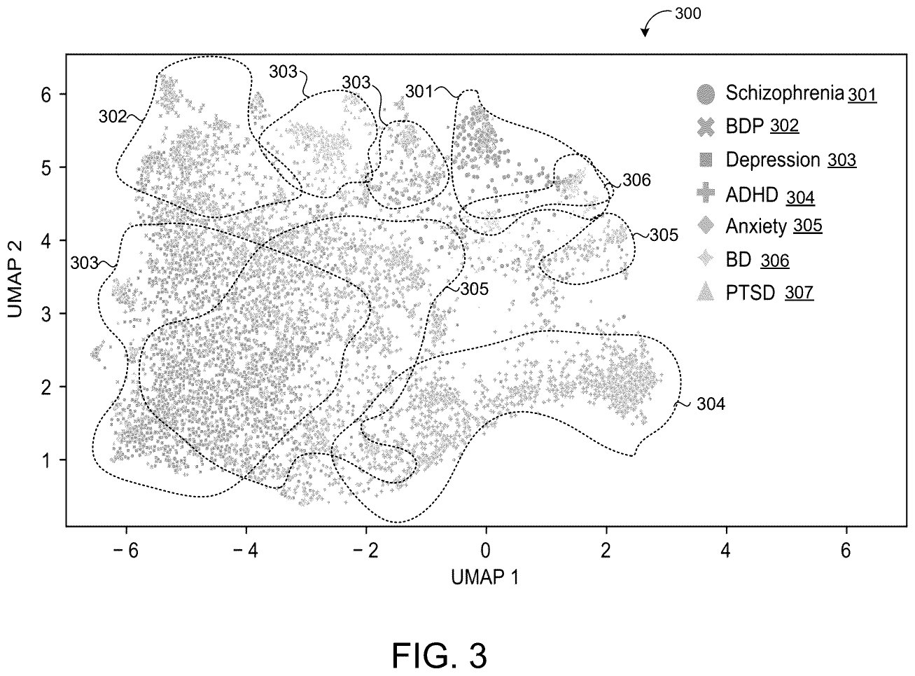

A projector 190, such as a Uniform Manifold Approximation and Projection (UMAP) algorithm, may receive each LLM embedding 162 generated by the LLM 160 and generate a two-dimensional visualization 300 (FIG. 3) that represents the LLM embeddings 162. The projector 190 may additionally receive the psychiatric disorder/condition label 104 paired with each textual utterance 110 input to the LLM 160 that indicates the respective psychiatric disorder/condition the textual utterance 110 in the respective textual utterance set 102A-N originated from. Here, the application 105 may instruct the user interface 170 to display, on a screen 112 in communication with the user device 110, the two-dimensional visualization 30 representing the LLM embeddings 162 projected onto a two-dimensional space.

FIG. 3 illustrates a plot of the two-dimensional visualization 30 representing the LLM embeddings 162 projected onto the two-dimensional embedding space. Here, the projector 190 may graphically distinguish LLM embeddings 162 from one another based on their paired psychiatric disorder/condition label 104. For instance, and continuing with the example, LLM embeddings 162 originating from the subreddit class for schizophrenia (r/schizophrenia) 301, LLM embeddings 162 originating from the subreddit class (r/bpd) 302 for borderline personality disorder, LLM embeddings 162 originating from the subreddit class (r/depression) 303 for depression, LLM embeddings 162 originating from the subreddit class (r/adhd) 304 for ADHD, LLM embeddings 162 originating from the subreddit class (r/anxiety) 305 for anxiety, LLM embeddings 162 originating from the subreddit class (r/ptsd) 307 for PTSD, and LLM embeddings 162 originating from the subreddit class (r/bipolarreddit) 306 for bipolar disorder.

The visualization 30 representing the LLM embeddings 162 projected onto the two-dimensional space reveals a number of qualitative insights. For instance, the LLM embeddings 162 originating from the posts of the r/anxiety 305 subreddit class are projected in the middle of the two-dimensional space, thereby neighboring the LLM embeddings 162 originating from the posts of the other subreddit classes. This insight may suggest that individuals with anxiety may use language that has some presence in all of the other psychiatric disorder/conditions. Another qualitative insight reveals that LLM embeddings 162 originating from the subreddit classes r/ptsd 307, r/bipolarredit 306, r/adhd 304, r/bpd 302, and r/schizophrenia 301 each form distinct clusters within the two-dimensional space represented by the visualization 30, while the other two subreddit classes r/anxiety 305 and r/depression 303) have more overlapping point clouds within the two-dimensional space of the visualization 30. Thus, the three subreddit classes with the overlapping point clouds suggest a greater linguistic similarity compared to the other subreddit classes.

The classification model 150 and associated softmax objective function 155 are configured to simultaneously predict the psychiatric classification labels 180 of the different psychiatric disorders/conditions based on the LLM embeddings 162 derived/generated by the LLM 160 from the textual utterances 110 originating from the plurality of common psychiatric disorders/conditions. The classification model 150 processes each LLM embedding 162 to predict a probability distribution over possible psychiatric classification labels 154, while the softmax objective function 155 selects the psychiatric classification label having the highest probability from the probability distribution over possible psychiatric classification labels 154 as the predicted psychiatric classification label 180. The softmax objective function 155 may be implemented as a dedicated layer of the classification model 150 or may be separate from the classification model 150.

In some examples, the classification model 150 includes a multiclass classifier that employs Extreme Gradient Boosting (XGBoost), which includes a supervised learning algorithm trained to accurately predict a target variable by combing an ensemble of estimates from a set of simpler and weaker models. Specifically, for each corresponding LLM embedding 162 generated by the LLM 160, the classification model 150 is trained to learn how to predict the corresponding psychiatric disorder/condition label 104 paired with the respectively textual utterance 110 from which the corresponding LLM embedding 162 was derived. Here, the corresponding psychiatric disorder/condition label 104 serves as a ground-truth label indicating the respective psychiatric disorder/condition from which the corresponding textual utterance 110 originated. In some examples, a respective training loss is derived for each psychiatric disorder/condition based on the psychiatric classification labels 180 predicted for the textual utterances 110 from the respective cleaned textual utterance set originating from the psychiatric disorder/condition and the corresponding psychiatric disorder/condition label 104 paired with the textual utterances that indicates the psychiatric disorder condition. Additionally or alternatively, each respective training loss may be derived based on the probability distribution over possible psychiatric classification labels 155 determined by the classification model for the textual utterances 110 originating the respective psychiatric condition and the corresponding psychiatric disorder/condition label 104 paired with the textual utterances that indicates the psychiatric disorder condition. As such, the classification model 150 may be trained based on the training losses derived for the multiple common psychiatric or neurological disorders. Notably, the classification model 150 may receive frequency information 152 associated with the number of textual utterances 110 in each respective cleaned textual utterance set 102A_{CLEAN}-N_{CLEAN}. Here, the classification model 150 may use the frequency information 152 during training to account for potential biases due to class imbalance by assigning weights to the LLM embeddings 162 associated with each respective psychiatric or neurological disorder/condition that are inversely proportional to the number of textual utterances in each respective cleaned textual utterance set. In some examples, the classification model 150 receives the table 200 of FIG. 2 corresponding to the cleaned textual utterance sets 102A_{CLEAN}-N_{CLEAN} and derives the frequency information 152 for each respective cleaned textual utterance set from the column that indicates the number of posts/textual utterances 110 in the respective textual utterance set for each respective psychiatric or neurological disorder/condition.

With continued reference to FIG. 1, the objective softmax function 155 outputs the psychiatric classification label 180 for each corresponding textual utterance 110 from each respective cleaned textual utterance set 102A_{CLEAN}-N_{CLEAN}. The user interface 170 may output the psychiatric classification label 180 from the user device 110 or another device as a message or notification. The message or notification may include a graphic/text displayed on the screen 112 of the user device 100 that conveys the psychiatric classification label 180. After the classification model 150 is trained, the classification model 150 may be employed during inference to predict a psychiatric classification label 180 from an LLM embedding 162 generated by the LLM 160 from a textual utterance that is not paired a corresponding psychiatric disorder/condition label 104 indicating the respective psychiatric disorder/condition from which the textual utterance originated.

In some examples, the psychiatric classification label 180 predicted for each corresponding textual utterance 110 is annotated with the psychiatric disorder/condition label 104 that indicates the respective psychiatric disorder/condition the corresponding textual utterance 110 originated from. Here, an evaluator 195 may use the psychiatric disorder/condition label 104 as a ground truth for determining one or more evaluation metrics associated with the psychiatric classification label 180 that was predicted for each corresponding textual utterance 110 in each respective cleaned textual utterance set 102A_{CLEAN}-N_{CLEAN}. For instance, the evaluator 195 may determine evaluation metrics 196 such as precision, recall, and F1 scores. The application 105 may instruct the user interface 170 to provide one or more of the evaluation metrics 196 for output from the user device 110 and/or another device. The evaluator 195 may similarly determine the respective training losses for the psychiatric disorders/conditions based on the psychiatric classification labels 180 and the psychiatric disorder/condition labels 104.

Continuing with the example, table 2 below shows evaluation metrics 196 of precision, recall, and F1 scores for the classification model 150 based on the psychiatric classification label 180 that was predicted for each corresponding textual utterance 110 (e.g., online forum post, social media post, etc.) originating from one of the subreddit classes each related to a respective one of the psychiatric disorder/conditions.

TABLE 2

| | | | f1 |
|---|---|---|---|
| Subreddit | Precision | Recall | score |
| r/adhd | 0.86 | 0.86 | 0.86 |
| r/anxiety | 0.66 | 0.69 | 0.67 |
| r/bipolarredit | 0.68 | 0.50 | 0.58 |
| r/bpd | 0.65 | 0.61 | 0.63 |
| r/depression | 0.73 | 0.81 | 0.77 |
| r/ptsd | 0.78 | 0.63 | 0.70 |
| r/schizophrenia | 0.76 | 0.62 | 0.69 |

Table 2 shows that across all subreddit classes related to the different psychiatric disorders/conditions, the evaluator 195 determined evaluation metrics for the weighted average precision, recall, and F1 scores for the psychiatric classification labels 180 predicted by the classification model 150 using a 0.5 threshold to be 0.73, 0.68, and 0.70, respectively. Additionally, the evaluator 195 measured the overall accuracy of the classification model 150 to be 0.73. At the individual classification level for the classification labels 180 predicted for each of the plurality of psychiatric disorders/conditions, the classification model 150 performed best at predicting classification labels 180 for ADHD and depression with f1 scores equal to 0.82 and 0.74, respectively. By contrast, the classification model 150 performed the worst at predicting classification labels 180 for borderline personality disorder and bipolar disorder with f1 scores equal to 0.48 and 0.50, respectively. As such, these evaluation metrics 196 indicate that the classification model 150 demonstrated moderate predictive performance in identifying the correct subreddit class related to each respective psychiatric disorder/condition from which each textual utterance 110 (e.g., online forum post, social media post, etc.) originated.

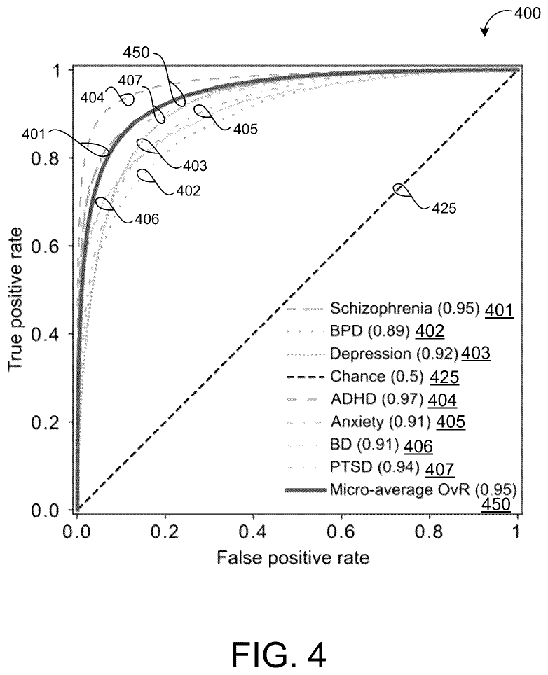

Additionally, for each corresponding cleaned textual utterance set 102A_{CLEAN}-N_{CLEAN} including respective textual utterances 110 corresponding to online forum posts originating from a respective one of the subreddit classes, the evaluator 195 may perform a one-vs-rest classification task to estimate an area under the curve (AUC) values based on probabilities of the classification labels 180 predicted for the respective textual utterances 110 in the corresponding cleaned textual utterance set. FIG. 4 shows an example plot 400 of receiver operating characteristic (ROC) curves and AUC values estimated for each of the subreddit classes related to the psychiatric disorder/conditions. The x-axis denotes a false positive rate and the y-axis denotes a true positive rate. For instance, and continuing with the example, the example plot 400 shows an ROC curve 401 estimated for the subreddit class for schizophrenia (r/schizophrenia), an ROC curve 402 estimated for the subreddit class (r/bpd) 402 for borderline personality disorder, an ROC curve 403 estimated for the subreddit class (r/depression) for depression, an ROC curve 404 estimated for the subreddit class (r/adhd) 404 for ADHD, an ROC curve 405 estimated for the subreddit class (r/anxiety) for anxiety, an ROC curve 406 estimated for the subreddit class (r/bipolarreddit) for bipolar disorder, and an ROC curve 407 estimated for the subreddit class (r/ptsd) for PTSD. Dashed line 425 represents a chancel level for an AUC value equal to 0.5. The example plot 400 also depicts a micro-average OvR curve associated with an AUC value equal to 0.95. The relatively high AUC values ranging from 0.89-0.97 indicate that textual utterances 110 (e.g., posts) originating from each respective subreddit class are highly distinguishable from the textual utterances 110 originating from each other subreddit class. Notably, the subreddit class r/adhd for ADHD has the highest AUC value of 0.97 suggesting that ADHD is topically most dissimilar from the other psychiatric disorders/conditions related to the other subreddit classes. On the other hand, the subreddit class r/bpd for borderline personality disorder has the lowest AUC value of 0.89 indicating that the textual utterances 110 (e.g., posts) originating from the subreddit class r/bpd share linguistic features with many of the textual utterances 110 originating from the other subreddit classes.

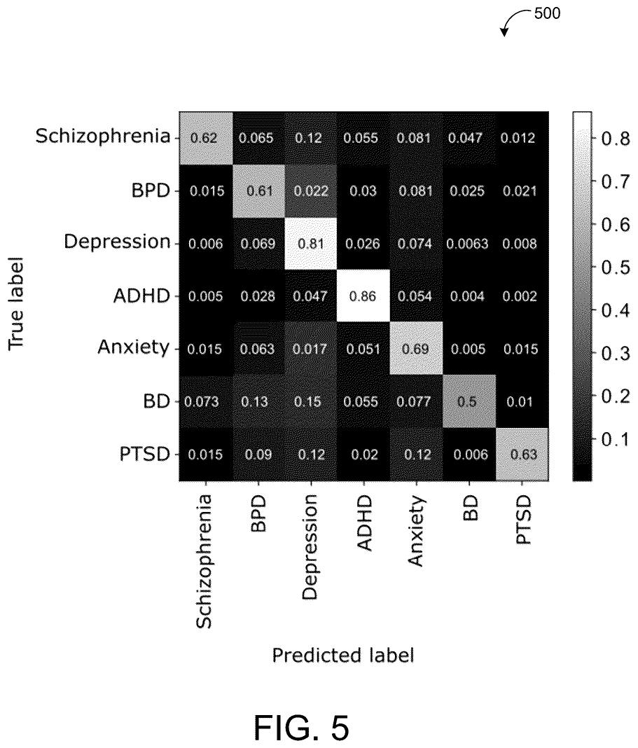

Referring to FIGS. 1 and 5, in some examples, the evaluator 195 is configured to compute a confusion matrix 500 that assesses the performance of the classification model 150 at the individual classification/category level and across pairs of classifications/categories. FIG. 5 shows a multi-class confusion matrix 500 based on the classification labels 180 predicted for the respective textual utterances 110 originating from each respective subreddit class and the corresponding psychiatric/disorder labels 104 paired with the textual utterances 110 to serve as ground-truth labels. Here, the x-axis denotes the classification labels 180 predicted by the classification model 150 for each of the subreddit classes and the y-axis denotes the psychiatric-disorder labels 104 that serve as ground-truth labels. The values within each box of the confusion matrix represent confusion rates of correct and incorrect classification for each subreddit class predicted by the classification model 150, normalized by the total number of posts (see Table 200 of FIG. 2) in each subreddit class. The confusion matrix 500 reveals the following pairs of subreddit classes to be the four most common true-predicted classification confusions made by the classification model 150: r/bpd-r/depression, r/anxiety-r/depression, r/bipolarreddit-r/depression, r/bipolarreddit-r/bpd. Notably, the classification model 150 often misclassifying textual utterances/posts as originating from the subreddit class r/depression for depression may result from the textual utterances/posts actually originating from the subreddit classes r/bpd, r/anxiety, and r/bipolarreddit using language that is more similar to the textual utterances/posts actually originating from the subreddit class r/depression. Interestingly, the confusion rates between the subreddit classes r/bpd, r/bipolarreddit, and r/anxiety are all less than 0.13, suggesting that linguistic overlap between these subreddit classes is less than the confusion rates with the subreddit class r/depression. However, these confusion rates may be attributed to the overrepresentation of the number of textual utterances/posts originating from the subreddit class r/depression (e.g., 11,513 textual utterances/posts in the third cleaned textual utterance set 102C_{CLEAN} corresponding to the subreddit class r/depression) that is incompletely offset by the class weighting when training the classification model 150.

In some implementations, for each respective cleaned textual utterance set 102A_{CLEAN}-N_{CLEAN}, a respective centroid value within the high-dimensional embedding space 300 (FIG. 3) is derived from the values of the LLM embeddings 162 that were derived from the respective set of multiple textual utterances 110 within the respective cleaned textual utterance set that originate from individuals having the respective one of the psychiatric disorders/conditions among the plurality of common psychiatric disorders/conditions. As such, each cleaned textual utterance set 102A_{CLEAN}-N_{CLEAN} may be assigned a respective centroid value. In some configurations, classification labels are predicted for the LLM embeddings based on a similarity score between the values of the LLM embeddings and the values of the respective centroid values assigned to the each psychiatric disorder/condition.

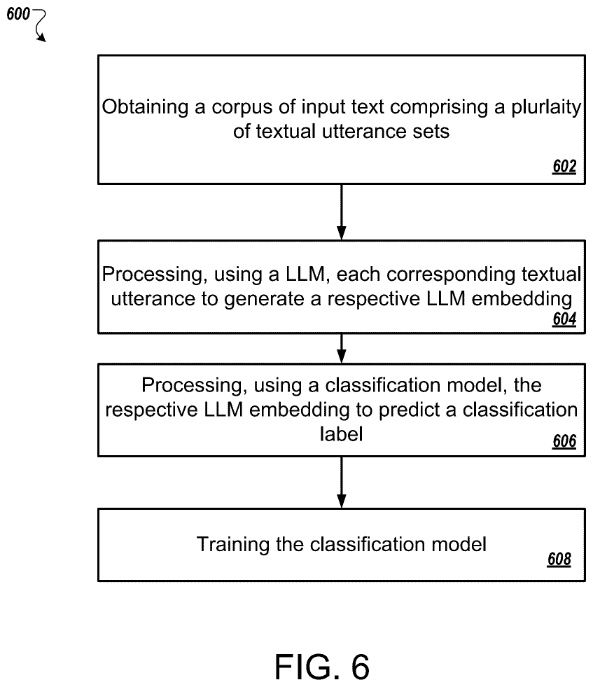

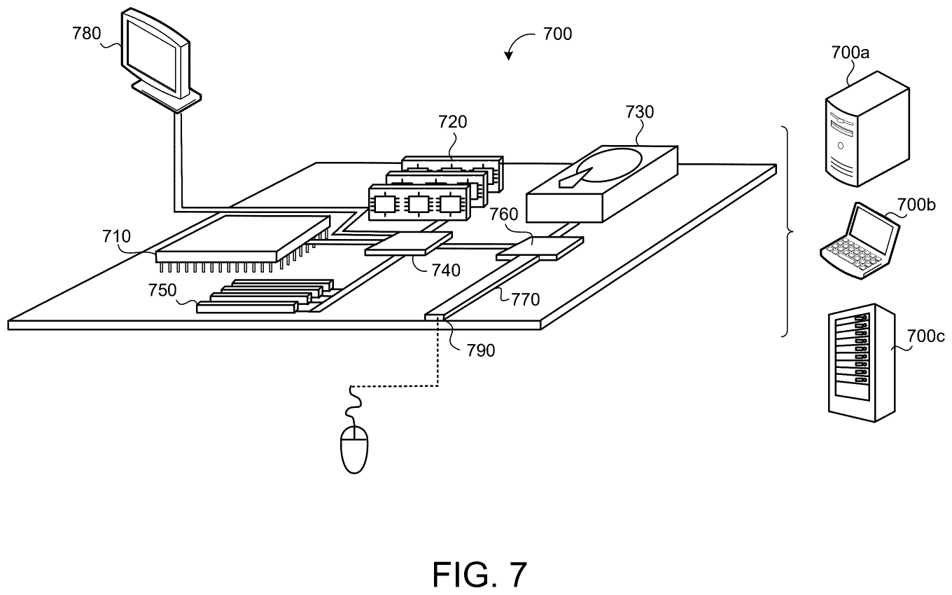

FIG. 6 is a flowchart of an example arrangement of operations for a method 600 of predicting classification labels for psychiatric disorders/conditions. The operations for the method 600 execute on data processing hardware 710 (FIG. 7) based on instructions stored on memory hardware 720 (FIG. 7) in communication with the data processing hardware 710. The data processing hardware 710 may include the data processing hardware 113 of the user device 110 and/or the data processing hardware 123 of the remote computing system 120. The memory hardware 720 may include the memory hardware 114 of the user device 110 and/or the memory hardware 124 of the remote computing system 120. At operation 602, the method 600 includes obtaining a corpus of input text 102 including a plurality of textual utterance sets 102A-N. Each textual utterance set from the plurality of textual utterance sets includes a respective set of multiple textual utterances 110 originating from a respective one of multiple disorders/conditions.

Operations 604 and 606 are performed for each corresponding textual utterance 110 from each respective textual utterance set. At operation 604, the method includes processing, using a large language model (LLM) 160, the corresponding textual utterance 110 to generate a respective LLM embedding 162 that represents the corresponding textual utterance 110 in a high dimensional embedding space 30. The corresponding textual utterance is paired with a corresponding disorder/condition label 104 indicating the respective disorder/condition from which the corresponding textual utterance originated. At operation 606, the method 600 includes processing, using a classification model 150, the respective LLM embedding 162 to predict a classification label 180 for the corresponding textual utterance 110. The predicted psychiatric classification label 180 includes one of the multiple disorders/conditions.

At operation 608, the method 600 includes training the classification model based on the classification label predicted for each corresponding textual utterance and the corresponding disorder/condition label paired with each corresponding textual utterance.

A software application (i.e., a software resource) may refer to computer software that causes a computing device to perform a task. In some examples, a software application may be referred to as an “application,” an “app,” or a “program.” Example applications include, but are not limited to, system diagnostic applications, system management applications, system maintenance applications, word processing applications, spreadsheet applications, messaging applications, media streaming applications, social networking applications, and gaming applications.

The non-transitory memory may be physical devices used to store programs (e.g., sequences of instructions) or data (e.g., program state information) on a temporary or permanent basis for use by a computing device. The non-transitory memory may be volatile and/or non-volatile addressable semiconductor memory. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

FIG. 7 is schematic view of an example computing device 700 that may be used to implement the systems and methods described in this document. The computing device 700 is intended to represent various forms of digital computers, such as laptops, desktops, workstations, personal digital assistants, servers, blade servers, mainframes, and other appropriate computers. The components shown here, their connections and relationships, and their functions, are meant to be exemplary only, and are not meant to limit implementations of the inventions described and/or claimed in this document.

The computing device 700 includes a processor 710, memory 720, a storage device 730, a high-speed interface/controller 740 connecting to the memory 720 and high-speed expansion ports 750, and a low speed interface/controller 760 connecting to a low speed bus 770 and a storage device 730. Each of the components 710, 720, 730, 740, 750, and 760, are interconnected using various busses, and may be mounted on a common motherboard or in other manners as appropriate. The processor 710 can process instructions for execution within the computing device 700, including instructions stored in the memory 720 or on the storage device 730 to display graphical information for a graphical user interface (GUI) on an external input/output device, such as display 780 coupled to high speed interface 740. In other implementations, multiple processors and/or multiple buses may be used, as appropriate, along with multiple memories and types of memory. Also, multiple computing devices 700 may be connected, with each device providing portions of the necessary operations (e.g., as a server bank, a group of blade servers, or a multi-processor system).

The memory 720 stores information non-transitorily within the computing device 700. The memory 720 may be a computer-readable medium, a volatile memory unit(s), or non-volatile memory unit(s). The non-transitory memory 720 may be physical devices used to store programs (e.g., sequences of instructions) or data (e.g., program state information) on a temporary or permanent basis for use by the computing device 700. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

The storage device 730 is capable of providing mass storage for the computing device 700. In some implementations, the storage device 730 is a computer-readable medium. In various different implementations, the storage device 730 may be a floppy disk device, a hard disk device, an optical disk device, or a tape device, a flash memory or other similar solid state memory device, or an array of devices, including devices in a storage area network or other configurations. In additional implementations, a computer program product is tangibly embodied in an information carrier. The computer program product contains instructions that, when executed, perform one or more methods, such as those described above. The information carrier is a computer- or machine-readable medium, such as the memory 720, the storage device 730, or memory on processor 710.

The high speed controller 740 manages bandwidth-intensive operations for the computing device 700, while the low speed controller 760 manages lower bandwidth-intensive operations. Such allocation of duties is exemplary only. In some implementations, the high-speed controller 740 is coupled to the memory 720, the display 780 (e.g., through a graphics processor or accelerator), and to the high-speed expansion ports 750, which may accept various expansion cards (not shown). In some implementations, the low-speed controller 760 is coupled to the storage device 730 and a low-speed expansion port 790. The low-speed expansion port 790, which may include various communication ports (e.g., USB, Bluetooth, Ethernet, wireless Ethernet), may be coupled to one or more input/output devices, such as a keyboard, a pointing device, a scanner, or a networking device such as a switch or router, e.g., through a network adapter.

The computing device 700 may be implemented in a number of different forms, as shown in the figure. For example, it may be implemented as a standard server 700a or multiple times in a group of such servers 700a, as a laptop computer 700b, or as part of a rack server system 700c.

Various implementations of the systems and techniques described herein can be realized in digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A number of implementations have been described. Nevertheless, it will be understood that various modifications may be made without departing from the spirit and scope of the disclosure. Accordingly, other implementations are within the scope of the following claims.

What is claimed is:

1. A computer-implemented method executed on data processing hardware that causes the data processing hardware to perform operations comprising:
obtaining a corpus of input text comprising a plurality of textual utterance sets, each textual utterance set from the plurality of textual utterance sets comprising a respective set of multiple textual utterances originating from a respective one of multiple disorders/conditions;
for each corresponding textual utterance from each respective textual utterance set:
processing, using a large language model (LLM), the corresponding textual utterance to generate a respective LLM embedding that represents the corresponding textual utterance in a high dimensional embedding space, wherein the corresponding textual utterance is paired with a corresponding disorder/condition label indicating the respective disorder/condition from which the corresponding textual utterance originated; and
processing, using a classification model, the respective LLM embedding to predict a classification label for the corresponding textual utterance, the predicted classification label comprising one of the multiple disorders/conditions; and
based on the classification label predicted for each corresponding textual utterance and the corresponding disorder/condition label paired with each corresponding textual utterance, training the classification model.

2. The computer-implemented method of claim 1, wherein the operations further comprise:
receiving regular expression codes indicating a list of terms related to the multiple disorders/conditions; and
for each respective textual utterance set:
inspecting, using the regular expression codes, the textual utterances from the respective set of multiple textual utterances to identify any textual utterances containing one or more expressions related to the respective disorder/condition among the multiple disorders/conditions from which the respective set of multiple textual utterances originated; and
for each corresponding textual utterance in the respective set of multiple textual utterances identified as containing any of the one or more expressions related to the respective disorder/condition, modifying or removing the corresponding textual utterance from the respective textual utterance set.

3. The computer-implemented method of claim 1, wherein each textual utterance from each respective set of multiple textual utterances comprises one or more sentences.

4. The computer-implemented method of claim 1, wherein each textual utterance from each respective set of multiple textual utterances comprises a post from an individual to an online forum topic associated with the respective disorder/condition.

5. The computer-implemented method of claim 1, wherein the multiple disorders/conditions comprise multiple common psychiatric disorders/conditions.

6. The computer-implemented method of claim 5, wherein the multiple common psychiatric disorders/conditions comprise schizophrenia, borderline personality disorder, depression, attention deficit hyperactivity disorder, anxiety, post-traumatic stress disorder, and bipolar disorder.

7. The computer-implemented method of claim 5, wherein the multiple common psychiatric disorders/conditions are selected from the group consisting of schizophrenia, borderline personality disorder, depression, attention deficit hyperactivity disorder, anxiety, post-traumatic stress disorder, and bipolar disorder.

8. The computer-implemented method of claim 1, wherein the operations further comprise:
receiving frequency information associated with the number of textual utterances in each respective textual utterance set,
wherein training the classification model is further based on the frequency information.

9. The computer-implemented method of claim 8, wherein the classification model is trained based on the frequency information by assigning weights to the respective LLM embeddings generated for the textual utterances in each respective textual utterance set that are that are inversely proportional to the number of the textual utterances in each respective textual utterance set.

10. The computer-implemented method of claim 1, wherein the LLM is trained using representational instruction tuning.

11. The computer-implemented method of claim 10, wherein the LLM is further trained using generative instruction tuning.

12. The computer-implemented method of claim 1, wherein the LLM comprises a plurality of sliding window attention (SWA) layers.

13. The computer-implemented method of claim 1, wherein the LLM comprises a plurality of grouped-query attention (GQA) layers.

14. The computer-implemented method of claim 1, wherein, when processing the corresponding textual utterance to generate the respective LLM embedding, the LLM employs bidirectional attention followed by mean pooling of a final hidden state to generate the respective LLM embedding.

15. The computer-implemented method of claim 1, wherein the classification model comprises a multiclass classifier that employs extreme gradient boosting.

16. The computer-implemented method of claim 1, wherein the operations further comprise generating a two-dimensional visualization that represents the LLM embeddings projected in a two-dimensional embedding space.

17. The computer-implemented method of claim 1, wherein the operations further comprise, after training the classification model:
receiving a textual input of one or more sentences from an individual, the textual input not paired with any disorder/condition label that indicates the respective disorder/condition associated with the corresponding textual utterance;
processing, using the LLM, the textual input to generate a respective LLM embedding that represents the textual input in the high dimensional embedding space; and
processing, using the trained classification model, the respective LLM embedding to predict a classification label for the textual input.

18. A system comprising:
data processing hardware; and
memory hardware in communication with the data processing hardware and storing instructions that when executed by the data processing hardware causes the data processing hardware to perform operations comprising:
obtaining a corpus of input text comprising a plurality of textual utterance sets, each textual utterance set from the plurality of textual utterance sets comprising a respective set of multiple textual utterances originating from a respective one of multiple disorders/conditions;
for each corresponding textual utterance from each respective textual utterance set:
processing, using a large language model (LLM), the corresponding textual utterance to generate a respective LLM embedding that represents the corresponding textual utterance in a high dimensional embedding space, wherein the corresponding textual utterance is paired with a corresponding disorder/condition label indicating the respective disorder/condition from which the corresponding textual utterance originated; and
processing, using a classification model, the respective LLM embedding to predict a classification label for the corresponding textual utterance, the predicted classification label comprising one of the multiple disorders/conditions; and
based on the classification label predicted for each corresponding textual utterance and the corresponding disorder/condition label paired with each corresponding textual utterance, training the classification model.

19. The system of claim 18, wherein the operations further comprise:
receiving regular expression codes indicating a list of terms related to the multiple disorders/conditions; and
for each respective textual utterance set:
inspecting, using the regular expression codes, the textual utterances from the respective set of multiple textual utterances to identify any textual utterances containing one or more expressions related to the respective disorder/condition among the multiple disorders/conditions from which the respective set of multiple textual utterances originated; and
for each corresponding textual utterance in the respective set of multiple textual utterances identified as containing any of the one or more expressions related to the respective disorder/condition, modifying or removing the corresponding textual utterance from the respective textual utterance set.

20. The system of claim 18, wherein each textual utterance from each respective set of multiple textual utterances comprises one or more sentences.

21. The system of claim 18, wherein each textual utterance from each respective set of multiple textual utterances comprises a post from an individual to an online forum topic associated with the respective disorder/condition.

22. The system of claim 18, wherein the multiple disorders/conditions comprise multiple common psychiatric disorders/conditions.

23. The system of claim 22, wherein the multiple common psychiatric disorders/conditions comprise schizophrenia, borderline personality disorder, depression, attention deficit hyperactivity disorder, anxiety, post-traumatic stress disorder, and bipolar disorder.

24. The system of claim 22, wherein the multiple common psychiatric disorders/conditions are selected from the group consisting of schizophrenia, borderline personality disorder, depression, attention deficit hyperactivity disorder, anxiety, post-traumatic stress disorder, and bipolar disorder.

25. The system of claim 18, wherein the operations further comprise:
receiving frequency information associated with the number of textual utterances in each respective textual utterance set,
wherein training the classification model is further based on the frequency information.

26. The system of claim 25, wherein the classification model is trained based on the frequency information by assigning weights to the respective LLM embeddings generated for the textual utterances in each respective textual utterance set that are that are inversely proportional to the number of the textual utterances in each respective textual utterance set.

27. The system of claim 18, wherein the LLM is trained using representational instruction tuning.

28. The system of claim 27, wherein the LLM is further trained using generative instruction tuning.

29. The system of claim 18, wherein the LLM comprises a plurality of sliding window attention (SWA) layers.

30. The system of claim 18, wherein the LLM comprises a plurality of grouped-query attention (GQA) layers.

31. The system of claim 18, wherein, when processing the corresponding textual utterance to generate the respective LLM embedding, the LLM employs bidirectional attention followed by mean pooling of a final hidden state to generate the respective LLM embedding.

32. The system of claim 18, wherein the classification model comprises a multiclass classifier that employs extreme gradient boosting.

33. The system of claim 18, wherein the operations further comprise generating a two-dimensional visualization that represents the LLM embeddings projected in a two-dimensional embedding space.

34. The system of claim 18, wherein the operations further comprise, after training the classification model:
receiving a textual input of one or more sentences from an individual, the textual input not paired with any disorder/condition label that indicates the respective disorder/condition associated with the corresponding textual utterance;
processing, using the LLM, the textual input to generate a respective LLM embedding that represents the textual input in the high dimensional embedding space; and
processing, using the trained classification model, the respective LLM embedding to predict a classification label for the textual input.
